(19) United States
(12) Patent Application Publication
(10) Pub. No.: US 2024/0084237 A1
(43) Pub. Date: Mar. 14, 2024

(54) A DEVICE HAVING A SUPPORT STRUCTURE FOR CREATING A HOLLOW, THREE-DIMENSIONAL ORGANOIDS AND CULTURES

(71) Applicant: The Trustees of Indiana University, Bloomington, IN (US)

(72) Inventors: FENG GUO, Bloomington, IN (US); ZHENG AO, Bloomington, IN (US); HONGWEI CAI, Bloomington, IN (US)

(21) Appl. No.: 18/272,455

(22) PCT Filed: Jan. 13, 2022
(86) PCT No.: PCT/US22/12313

§ 371 Date: Jul. 14, 2023

Related U.S. Application Data
(60) Provisional application No. 63/137,787, filed on Jan. 15, 2021.

(51) Int. Cl. C12M 1/12; C12M 1/00; C12N 5/00

(52) U.S. Cl. CPC C12M 25/14; C12M 23/34; C12N 5/0062; C12N 2513/00

(57) ABSTRACT

Disclosed is an apparatus configured to support growth of a 3D, hollow organoid. The apparatus contains a support structure and an enclosure, wherein a number of cells positioned between the support structure and the enclosure can grow on/around the support structure to form the organoid. The support structure additionally contains openings such that a fluid within the structure can flow into the organoid. The support structure may also extend between two fluid reservoirs configured to supply a fluid to the structure and organoid. The entire apparatus may be formed from an additive manufacturing process.

### CROSS-REFERENCE TO RELATED APPLICATION

This application claims priority to U.S. Provisional Application No. 63/137,787, filed on Jan. 15, 2021, incorporated herein by reference in its entirety.

### FIELD OF THE DISCLOSURE

The devices and methods described herein generally relate to a device that includes a support structure for growing a hollow, three-dimensional (3D) organoid or culture.

### BACKGROUND

Neuroinflammation plays a central role in neural degenerative disease, autoimmune diseases as well as substance abuse. Microglia is the resident myeloid cell in the brain and orchestrates brain inflammatory responses. Homeostatic microglia survey the brain, perform synaptic pruning, facilitate synapse maturation, and phagocytizes debris. Upon stimulation by factors such as toll-like receptor (TLR) ligands, β-amyloid aggregates, complement factors, or opioids, microglia can shift to an activated state. Activated microglia can either secret pro-inflammatory cytokines such as TNF-α, IL-6, IL-1β, and IL-18 or anti-inflammatory cytokines such as IL-4, IL-10, and TGF-β depending on the dose, frequency, and context of the stimulation. Microglia can also shift between the two states throughout the course of disease progression. To further complicate this issue, microglia also communicates extensively with astrocytes and neurons to shape the local immune environment. Current neural inflammation studies are largely based on two-dimensional (2D) cultures which lack the complex brain microenvironment to preserve microglia native state and function, as well as the complex crosstalk between the neuron and glial cells. Animal models can reflect the native state of microglia, as well as their responses to perturbation. Yet, specific chemical and genetic manipulation of microglia in animal models is often challenging, inaccessible to real-time imaging, and cannot fully reflect human biology.

Human brain organoids are brain-like three-dimensional (3D) cultures derived from human pluripotent stem cells, and recapitulate native brain cellular components, cytoarchitecture, and electrophysiology. Yet, current brain organoid models lack mesoderm derived microglia cells, prohibiting its applications in neural-immune biology studies. To overcome this limitation, primary microglial cells or stem-cell-derived microglia can be introduced into brain organoids. Microglia incorporated into brain organoids can recapitulate ramified morphology as well as key microglia functions including phagocytosis, synapse pruning, and responses to inflammatory cues. More importantly, these 3D organoid-based models can also be derived from isogenic human induced pluripotent stem cells (iPSC) or genetically engineered stem cells to recapitulate key human disease-related phenotypes. For example, the Cho group has developed a tri-culture system with the neuron, astrocytes, and microglia to study their interactions in an Alzheimer's model. Tsai group derived microglia from iPSC carrying Alzheimer's disease-related APOE4 variant and introduced these cells into brain organoids to study their uptake of Aβ. Blurton-Jones group integrated iPSC derived microglia into brain organoids and demonstrated their responses to needle induced injury. Li group functionalized brain-region specific organoids with isogenic microglia to study the transcriptome difference between microglia incorporated into dorsal and ventral brain regions. However, these models still suffer from a variety of brain organoid size, extensive manipulation of organoids during microglia incorporation, and, most importantly, hypoxic center in the brain organoids which can cause stress that impact organoid maturation and microglia functions.

To reduce hypoxic centers inside brain organoids, facilitate neuron maturation, and improve reproducibility, efforts have been made to promote oxygen perfusion and medium exchange in organoid cultures. Lancaster group has adopted the air-liquid interface method to culture sectioned brain organoids to improve organoid viability and promote axon growth. Ming group has performed serial sections of brain organoids to limit their thickness to below 500 μm to reduce hypoxia. Park group has engineered vascular system into brain organoid to facilitate perfusion. However, these methods are highly skilled dependent, require laborious manipulation, and not amenable to high-throughput organoid fabrication and culture.

### SUMMARY

What is needed is an engineered model that promotes organoid uniformity, allows non-invasive and high-efficiency microglia incorporation, most importantly, reduces or eliminates the hypoxic center of an organoid (e.g., brain organoid) is highly desired to model neural-immune crosstalk under homeostatic and inflamed conditions. The present disclosure discusses an organoid or 3D culture fabrication method and culture device with air-liquid interface culture to minimize hypoxia. This microfluidic culture platform holds great potential for easy-to-operate, scalable, and hypoxia-free organoid culture, such as brain organoid culture.

The engineered hollow, tubular human organoid model of the present disclosure discusses allows for guided growth of the organoid surrounding a support structure, which can be formed by a 3D printing process. The novel for forming an organoid has the following advantages: (1) the device and resulting organoid provides perfusion of medium and oxygen from both outside and an embedded hollow lumen in the center, limiting the distance between medium and any cells within the organoid culture to a distance <500 μm; (2) the device and resulting organoid can be perfused with the constant flow using a standard rocking platform, further improving medium and oxygen perfusion; (3) the device and resulting organoid allow simple and effective perfusion to incorporate microglia cells without any invasive injection or manipulation, preserving the native, homeostatic status of microglia cells; (4) the device and resulting organoid allow easy, cost-effective standardization of organoid culture to minimize variation which is also scalable and robust; and the device and tubular culture method can be easily adapted to other 3D cultures, such as intestinal and tumor organoids.

A device for supporting growth of a three dimensional tubular organoid or 3D culture of the present disclosure comprises: a support structure having a wall and a lumen extending from a first end to a second end, wherein the wall comprises a plurality of openings extending therethrough; an enclosure configured to extend at least partially around the support structure and support a plurality of cells disposed between the enclosure and the support structure; and a first reservoir fluidly coupled to at least one of the ends of the support structure.

The device of the previous paragraph, wherein the enclosure is configured to be removed from extending at least partially around the support structure.

The device of any of the previous paragraphs, wherein the plurality of openings comprise at least one of holes, pores, slots, mesh, and semi-permeable membranes.

The device of any of the previous paragraphs, wherein the plurality of openings have a size from about 1 micron to about 1 cm.

The device of any of the previous paragraphs, wherein the plurality of openings have a size of about 250 micron.

The device of any of the previous paragraphs, wherein the wall has a size from about 0.5 microns to about 2 millimeters.

The device of any of the previous paragraphs, wherein the wall has a size of about 1 micron to about 500 microns.

The device of any of the previous paragraphs, wherein the support structure is formed from an additive manufacturing process.

The device of any of the previous paragraphs, wherein the support structure is composed of a biocompatible polymer.

The device of any of the previous paragraphs, further comprising a second reservoir, wherein the first reservoir is fluidly coupled to the first end of the support structure and the second reservoir is fluidly coupled to the second end of the support structure.

The device of any of the previous paragraphs, further comprising a fluid agent within the first and second reservoirs, the fluid comprising at least one of a medium, cells, and therapeutic agents.

The device of any of the previous paragraphs, wherein the fluid agent is configured to contact at least a portion of the 3D organoid through the permeable features.

The device of any of the previous paragraphs, wherein the system is configured to be positioned within a standard well plate.

A method of growing a three dimensional hollow organoid of the present disclosure comprises: providing a device comprising: a support structure having a wall and a lumen extending from a first end to a second end of the lumen, wherein the wall comprises a plurality of openings extending therethrough; an enclosure configured to extend at least partially around the support structure and support a plurality of cells disposed between the enclosure and the support structure; a first reservoir fluidly coupled to the first end of the support structure; and a second reservoir fluidly coupled to the second end of the support structure; positioning the plurality of cells between the support structure and the enclosure; and providing at least one fluid into the first reservoir or the second reservoir and allowing the at least one fluid to flow into the lumen of the support structure and through the openings of the support structure, wherein the at least one fluid contacts the cells, thereby facilitating growth of the cells into a three dimensional hollow organoid surrounding the support structure.

The method of the previous paragraph, further comprising the step of treating the three dimensional organoid with an active agent.

The method of any of the previous paragraphs, further comprising rocking the device about an axis perpendicular to a length of the support structure, wherein the length is defined to be from the first end to the second end of the support structure.

The method of any of the previous paragraphs, wherein the device is composed of a biocompatible polymer.

The method of any of the previous paragraphs, wherein the support structure is tubular.

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a perspective view of an example of an organoid support comprising a hollow, permeable support structure disposed between two reservoirs according to an embodiment of the present disclosure;

FIG. 2 is a cross-sectional, perspective view of the hollow, permeable organoid support structure of FIG. 1 extending from a reservoir;

FIG. 3 is a cross-sectional view of the organoid support illustrating the hollow, permeable organoid support structure disposed between and fluidly coupled to the reservoirs of FIG. 1;

FIG. 4 is a perspective view of an example of an enclosure that is configured to surround the hollow, permeable organoid support structure according to an embodiment of the present disclosure;

FIG. 5 is a flowchart for an example of a method for growing a hollow, tubular organoid according to an embodiment of the present disclosure;

FIG. 6 illustrates a 3D hollow, permeable organoid according to an embodiment of the present disclosure;

FIG. 7 is a cross-sectional view of the hollow, permeable organoid of FIG. 6;

FIG. 8 illustrates the hollow, tubular organoid support structure of FIG. 1 with a fluid flowing through the lumen of the structure from one reservoir to the other reservoir;

FIG. 9 is a plot of the hollow, permeable, tubular organoid viability over time;

FIG. 10 is a plot of hypoxia intensity as a function of distance into the hollow, permeable, tubular organoid; and

FIG. 11 is an experimental example of organoid growth on a hollow, tubular organoid support structure according to an embodiment of the present disclosure.

It should be understood that the drawings and replicas of the photographs are not necessarily to scale. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

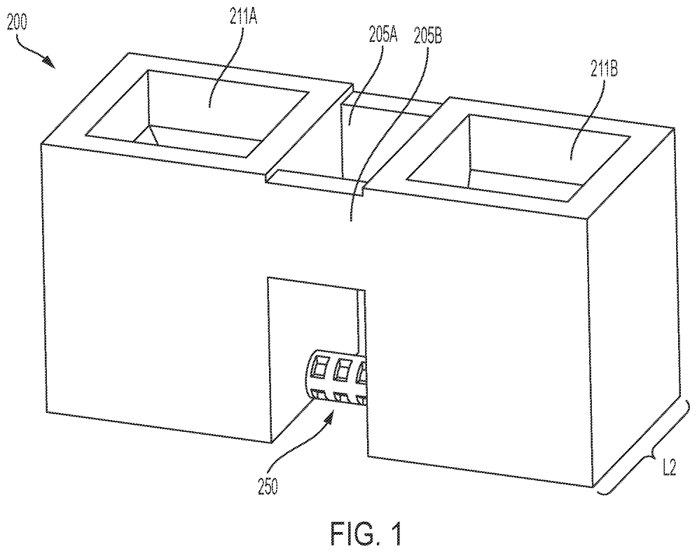

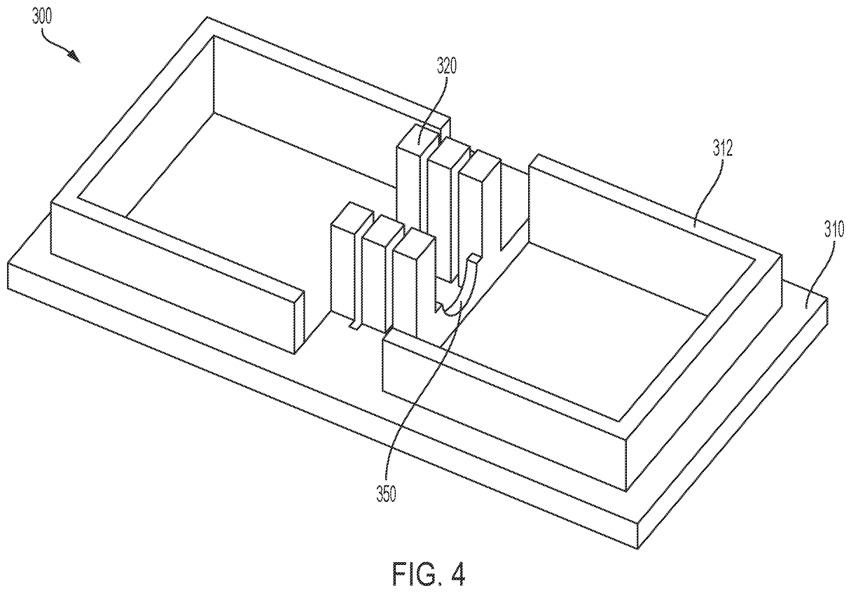

Referring to FIG. 1, there is depicted a supporting device 200 comprising a hollow, permeable support structure 250 disposed between two reservoirs 211A, 211B. Referring to FIG. 4, there is depicted an enclosure 300 a portion of which is configured to surround the hollow, permeable support structure 250. The supporting device 200 is combined with the enclosure 300 to form a cell growth device 100. For example, the enclosure 300 may be disposed under the supporting device 200 such that the supporting device 200 sits on top of above the enclosure 300, thereby creating a gap between the support structure 250 and the enclosing members 320 and/or support interface 350 of the enclosure 300 discussed in more detail below. The cell growth device 100, which may also be referred to as a chip, is generally configured to support the growth of a number of cells disposed in the gap between the support structure 250 and the enclosing members 320 and/or support interface 350. In an exemplary embodiment, cell growth device 100 is configured to receive a number of cells in the gap between the support structure 250 and the enclosing members 320 and/or support interface 350 and facilitate growth of those cells in the gap into a hollow, 3D organoid, such as a hollow tubular 3D organoid.

As shown in FIG. 1, supporting device 200 may comprise at least two reservoirs 211A, 211B and a support structure 250 disposed therebetween and coupling and/or fluidly coupling the reservoirs 211A, 211B. It is to be understood that features described with “#A, B” notation are similar in structure and function, and differ in location within the various embodiments. While typically two features are illustrated using the A, B notation, it should be understood that any number of features may be used, including one and more than two.

In the illustrated embodiment, in addition to the support structure 250 extending between and coupling the reservoirs 211A, 211B together, reservoirs 211A and 211 B are also coupled together through walls 205A and 205B. Walls 205A, 205B may provide structural support within supporting device 200. Walls 205A, 205B may also be configured to interact with enclosure 300. For example, walls 205A, 205B may abut, interlock with, couple to, or inhibit the movement of enclosure 300. In some embodiments, supporting device 200 may not comprise any connecting walls 205A, 205B and reservoirs may only be coupled through support structure 250. The walls 205A, 205B may be any shape or feature desired to provide support within supporting device 200 (e.g. pillars, arches, columns, blocks, etc.), and may comprise any number of structures. Walls 205A, 205B and any of the other walls within cell growth device 100 including but not limited to reservoir walls, support structure walls, enclosure walls, etc. may have any thickness. In some embodiments, the walls of cell growth device 100 have a thickness of about 0.5 microns (μm) to about 100 cm, from about 0.5 microns to about 2 mm, from about 0.5 microns to about 1 mm, from about 0.5 microns to about 500 microns, and about 1 micron to about 500 microns, from about 1 micron to about 400 microns, and from about 1 micron to about 300 microns.

Reservoirs 211A, 211B are configured to retain a fluid 400 (See FIG. 7). The fluid 400 is configured to flow from one reservoirs 211A, 211B to the other reservoir 211A, 211B through a lumen 255 in the support structure 250, which fluidly couples the reservoirs 211A, 211B to one another. The fluid 400 may be any fluid that is desired to flow through support structure 250 or through an organoid. Examples of a fluid 400 include, but are not limited to, growth medium, oxygen, air, aqueous solutions, organic solutions, dissolved gases, suspensions, therapeutic agents or pharmaceuticals, living cells, bacteria solutions, pathogenic solutions, and combinations thereof.

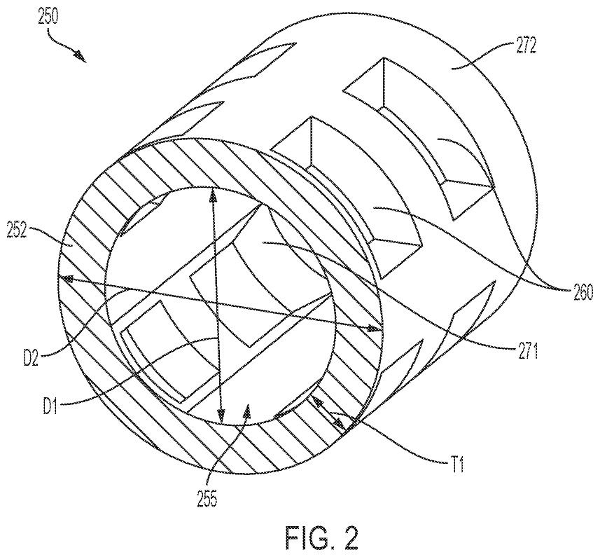

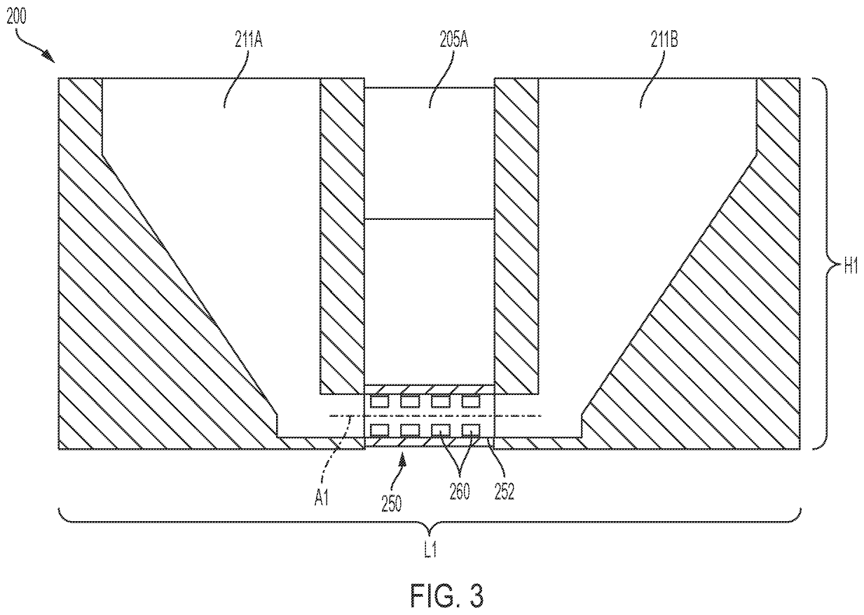

Referring now to FIGS. 2-3, support structure 250 may comprise a wall 252 and a lumen 255 or hollow interior extending from a first end to a second end, wherein the wall 252 comprises a plurality of openings 260 extending therethrough. The support structure 250 extends generally along a length or an axis A1 between the two reservoirs 211A, 211B, wherein the first end of the support structure 250 is coupled to one reservoir 211A or 211B and the second end is coupled to the other reservoir 211A, 211B. The wall 252 has an interior support surface 271, an exterior support surface 272, and a thickness T1. The thickness T1 of wall 252 may be from about 0.5 microns (μm) to about 5 cm, from about 0.5 microns to about 2 mm, from about 0.5 microns to about 1 mm, from about 0.5 microns to about 500 microns, and about 1 micron to about 500 microns, from about 1 micron to about 400 microns, and from about 1 micron to about 300 microns

In the illustrated embodiment, support structure 250 is shown as a hollow, tubular member with an inner diameter of D1 and an outer diameter of D2, but in other embodiments support structure 250 may be any shape or size suitable to support growth of cells, and may comprise any shape or size channel or throughput within. Support structure 250 may have multiple channels or lumens within any shaped structural support wall, and may extend between any number of reservoirs. For example, cross sections of support structure 250 (similar to FIG. 3) may be shaped as any polygon or closed figure and may comprise rounded edges. Additionally, support structure 250 may be comprise a number of individual support structures extending in multiple directions, such as forming a lattice, cross, or a Y-shape. As is described in additional detail herein, support structure 250 is configured to support a number of cells.

As mentioned above, the support structure 250 is fluidly coupled to reservoirs 211A, 211B such that a fluid may travel between the reservoirs through the interior or lumen 255 of support structure 250. Support structure 250 comprises a number of permeable features or openings 260 which are configured to allow some of the fluid that flows through support structure 250 from one end to the other end to also flow through and out of the openings 260 of the wall 252. Examples of openings 260 include, but are not limited to, holes, pores, slots, mesh, semi-permeable membranes, filters, channels. In some embodiments, support structure 250 creates or is composed of a permeable or semi-permeable material such that the entirety of support structure 250 is configured to allow a fluid to move outwardly through its wall 252 from the lumen and the inner surface of the wall. In embodiments where openings 260 are holes, slits, or other general open features, they may have a size from about 1 micron to about 2 cm, from about 1 micron to about 1 cm, from about 1 micron to about 500 microns, from about 1 micron to about 300 microns, from about 100 microns to about 400 microns, about 100 microns, about 200 microns, about 250 microns, about 300 microns, about 500 microns, or about 500 microns. In an exemplary embodiment, openings 260 are sized such that they are large enough that a fluid or an active compound may pass through openings 260, but small enough such that any cells that are placed on or around support structure 250 do not go through openings 260 into the interior of support structure 250. The size, number of and/or shape of the openings determine(s) the permeability of wall 252 of the support structure 250 and the surrounding organoid.

Referring now to FIG. 4, an enclosure 300 is shown. Enclosure 300 may comprise an enclosure base 310, interfacing walls 312, enclosing members 320, and a support interface 350. Enclosure 300 may be positioned below supporting device 200 and configured to at least partially surround support structure 250 to support and facilitate the growth of an organoid or other cell structure, while creating a gap between the support structure 250 and the enclosing members 320 and/or support interface 350 of the enclosure 300. Enclosure base 310 may be configured to rest on or couple to a surface when cell growth device 100 is fully assembled. Interfacing walls 312 are configured to interface with a bottom portion of supporting device 200 and to stabilize the overall structure of cell growth device 100 when assembled. Interfacing walls 312 are illustrated as walls, but may be any feature configured to interface with a portion of supporting device 200 including supports, locking members, detents, channels, protrusions, or other interfacing members. Enclosing members 320 are shown as a number of pillars or columns, which allows for the movement of fluid between them to reach support structure 250 and the cells supported thereon. In other embodiments, enclosing members 320 may be one continuous piece extending at least partially around support structure 250, or may be any other feature configured to at least partially contain a number of cells on or near support structure 250. Support interface 350 is configured to receive or otherwise interface with support structure 250, and accordingly is shown in the illustrated embodiments as a semi-circle or arc. In other embodiments support interface 350 may be any shape or size complimentary to the shape and size of support structure 250 such that enclosing members 320 may extend around support structure 250. Enclosure 300 may also be configured to limit the size of the organoid that grows on or around support structure 250 by altering the spacing between enclosing members 320, support interface 350, and support structure 250.

Enclosure 300 and supporting device 200 may also be configured such that an organoid that grows on or around support structure 250 may be observed or measured. For example, the space between walls 205A, 205B may be open such that the organoid may be viewed from above. In some embodiments, enclosure 300 has a clear portion or an opening allowing the organoid to be viewed from below. In other embodiments, supporting device 200 and/or enclosure 300 may be composed of a material permeable to electromagnetic radiation such that the organoid may be viewed from multiple angles. Some examples of measurement/observation include microscopy, electron scanning, IR, NMR, electrochemical tests, UV/Vis spectrometry, x-ray spectrometry, PCR, and dye testing methods.

Referring now to FIGS. 1-5, a method of growing an organoid is disclosed. As used herein, the term “organoid” may be used to describe a plurality of cells organized into any structure, such as organ-like 3D structures, 3D collections of cells, 3D tissue samples, 3D cultures, or any cellular culture in general. Exemplary embodiments of organoids include, but are not limited to, cerebral, intestinal, stomach, lingual, thyroid, hepatic, pancreatic, lung, kidney, cardiac, retinal, and cancerous organoids, as well as any other simplified organ structure, cell cultures, tissues, biological growths, and combinations thereof. The organoids of the present disclosure may be derived or grown from any source, including but not limited extracellular matrices, stem cells, tissue, embryonic bodies (EB), induced pluripotent stem cells, progenitor cells, or any other type of cell. Both organoids and the cells, cultures, or tissues from which they are grown may all be broadly referred to as a plurality of cells or a number of cells.

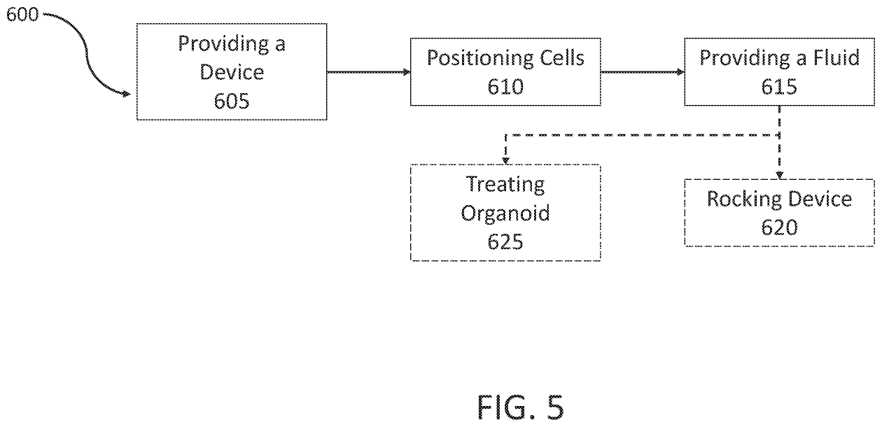

As shown in FIG. 5, there is shown an exemplary method 600 of growing an organoid using the device 100 of the present disclosure discussed above. The exemplary method 600 may include a providing a device step 605, a positioning cells step 610, and a providing a fluid step 615. The method 600 also optionally comprises a rocking device step 620 and a treating organoid step 625. Providing step 605 includes providing a device as disclosed herein, for example cell growth device 100. Positioning cells step 610 involves positioning a number of cells, which may be, for example, a cell mass, an embryonic body, or a tissue sample between support structure 250 and enclosing members 320. In an exemplary embodiment, cells may be placed on or in support interface 350 and supporting device 200 may then be positioned on enclosure 300 to secure cells between support structure 250 and enclosing members 320 such that the cells surround the support structure 250 without entering into the lumen 255. The providing a fluid step 615 includes providing a fluid 400 to the cells. The fluid 400 may be provided from one reservoir 211A, 211B through the lumen 255 and the openings 260 in the wall 252 of the support structure 250, or from the exterior of support structure 250. In an exemplary embodiment, the fluid 400 is configured to promote cell growth, specialization, conglomeration, or any combination of thereof. That is, the fluid contacts the cells and facilitates growth of the cells into a three dimensional hollow organoid surrounding the support structure 252. The steps 605, 610, and 615 are configured to cause a number of cells to aggregate and grow into a 3D culture or organoid around support structure 250. In the illustrated embodiment, the organoid grows into a 3D, hollow, tubular structure around support structure 250. Using cell growth device 100 to grow an organoid 500 allows the organoid to be relatively uniform between trials and allows ample proliferation of fluid 400 through the organoid, among other benefits.

The rocking device step 620 involves rocking the device to encourage flow of the fluid 400 through support structure 250 so that the fluid 400 interacts with the cells or organoid thereon. As is described in more detail herein, rocking may occur on a standard rocking device and may be in a direction perpendicular to the length of the support structure 250. The treating organoid step 625 involves treating either a collection of cells or a grown organoid or 3D culture with an active agent, such as a pharmaceutical or other cells. The treating step may be used to determine how certain cells or organoids respond to various stimuli, to encourage the growth of the organoid, or to otherwise affect the cells or organoid on or around support structure 250.

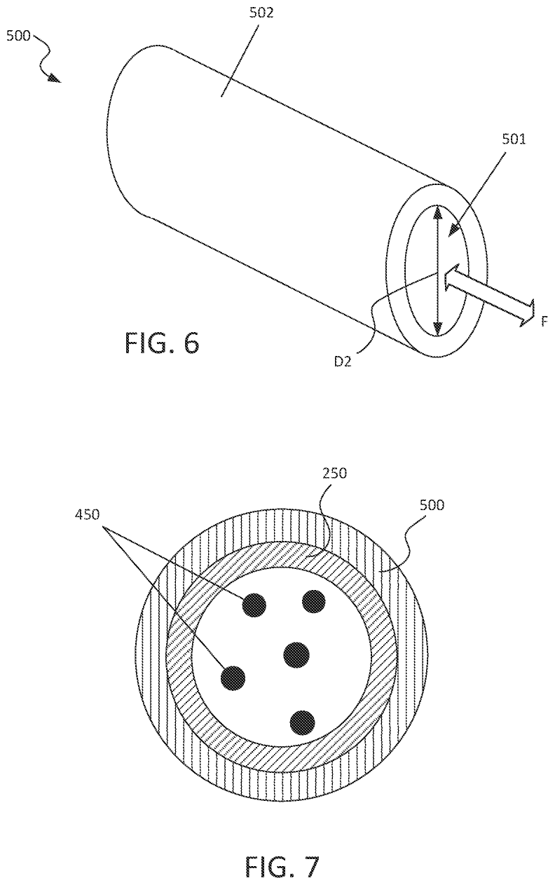

Referring to FIGS. 6-7, an embodiment of an organoid 500 is shown. In general, the organoid 500 is supported by support structure 250. Organoid 500 can be grown around any embodiment of support structure 250, and may grow to a corresponding shape. In the illustrated embodiments, organoid 500 is a tube with an outer surface 502 and an inner surface 501. The inner surface 501 grows around the outer surface 272 of support structure 250. Accordingly, the inner diameter of organoid 500 is approximately the same as the outer diameter of support structure 250, which in the illustrated embodiments is D2. Because organoid 500 is hollow, fluids can pass through the interior of organoid 500 as well as along the exterior of organoid 500, allowing diffusion into cells from inner surface 501 and outer surface 502. This configuration of organoid 500 reduces the likelihood of hypoxia, necrosis, lack of nutrients, or other issues found with organoids that lack an interior channel. As shown in FIG. 6, fluid 400 can pass through support structure 250 and organoid 500 as indicated by the direction of flow, F. Fluid 400 within support structure 250 can exit support structure 250 through openings 260 and diffuse into organoid 500. Fluid 400 may also come from sources other than reservoirs 211 A, B and come into contact with outer surface 502 of organoid 500. For example, the entirety of cell growth device 100 may be completely or partially submerged in fluid 400.

As shown in FIG. 7, fluid 400 may comprise active compounds or a fluid agent 450 that may flow though support structure 250 and diffuse into organoid 500 through openings 260. Examples of fluid agents 450 include, but are not limited to, growth medium, pharmaceuticals, enzymes, therapeutic agents, viruses, bacteria, hormones, myeloid cells, any other living cell, proteins, microparticles, nanoparticles, or any other compound that may interact with organoid 500. The fluid agents 450 may be mixed with fluid 400 within reservoirs 211 A, B.

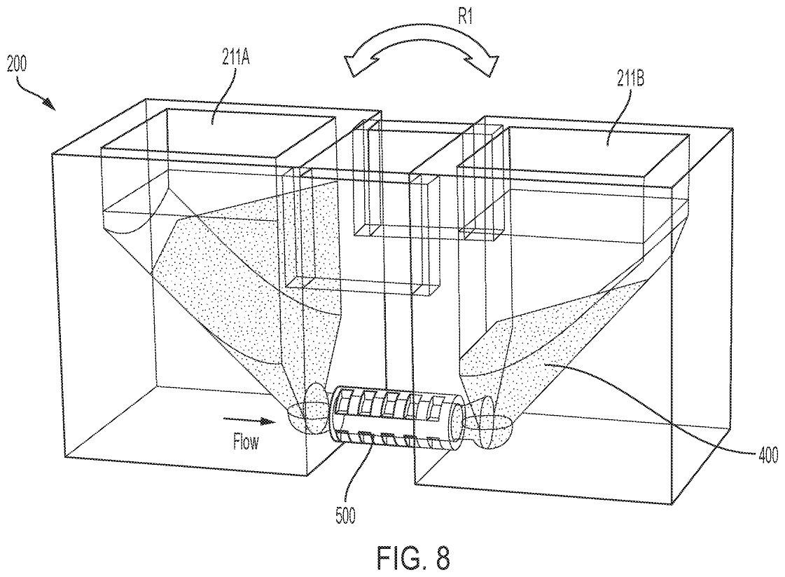

Referring now to FIG. 8, supporting device 200 is shown with an organoid 500 supported by support structure 250, and a fluid 400 within reservoirs 211A, 211B. The entire device may be rocked back and forth in the direction R1 to facilitate flow of fluid through support structure 250, providing adequate mixing of the fluid 400 and encouraging flow through support structure 250 and into organoid 500. The rocking motion generally occurs in a direction around an axis perpendicular to the length of support structure 250, or to axis A1 (FIG. 3). Such a rocking step may be incorporated into the method 600, and may be carried out on a rocking mechanism.

In an exemplary embodiment, cell growth device 100 is configured to be positioned within a well of a well plate, such as a 96 well plate. For example, cell growth device 100 may have a length, L1 and a depth L2 (See FIGS. 3 and 1) of less than about 7 mm, and may have a height H1 (See FIG. 3) less than about 12 mm. In other embodiments, cell growth device 100 may have any size suitable for growth of an organoid. The size of cell growth device 100 and support structure 250, as well as the relative positioning of enclosure 300 and support structure 250 may vary based on the shape and orientation of support structure 250, the type of organoid being grown, the type of cells being used, the type of fluid 400 and/or fluid agents 450 being used, the observational equipment used to observe the organoid 500, the number and position of reservoirs 211A, 211B, the surface or container used for holding cell growth device 100, and any other desirable structural motivators.

Any or all of the components of cell growth device 100 may be made from an additive manufacturing process. In additive manufacturing, hardware is directed to add material to create a structure. In an exemplary embodiment, cell growth device 100 is formed from a 3D printing process. Examples of additive processes that may be used to form some of any of the components of cell growth device 100 include, but are not limited to, powder bed fusion, binder jetting, directed energy deposition, material extrusion, sheet lamination, vat polymerization, sintering, direct laser melting, electron beam melting, stereolithography, and combinations thereof. Any or all of the components of cell growth device 100 may also be composed of any suitable material, including polymers, metals, composites, and organic substances. In some embodiments, cell growth device 100 is composed of a biocompatible polymer. Examples of materials that may be used in constructing any components of cell growth device 100 include, but are not limited to, methacrylate, bisphenol dimethacrylate, urethane dimethacrylate, polyurethanes, fluoropolymers, perfluoroether (PFA), fluorinated ethylene propylene (FEP), polyethylene terephthalate (PET), polyvinyl, polyvinyl alcohol (PVA), poly lactic acid (PLA), acrylonitrile butadiene styrene (ABS), polycarbonate, polypropylene, polyethylene, polytetrafluoroethylene (PTFE), polyether ether ketone (PEEK), cyclic olefins, polyglycolic acid (PGA), polycaprolactone (PCL), nylon, cellulose, polyamides, polystyrene, and combinations and copolymers thereof.

Additionally, various components of cell growth device 100 may be configured to have different material properties. For example, the interior surface 271 of support structure 250 may be composed of a hydrophobic material or coated with a hydrophobic coating to reduce the number of cells that may grow within support structure 250 or to prevent fluid agent 450 from adhering to the interior of support structure 250. Furthermore support structure 250 may be composed of a different material than the rest of supporting device 200 and/or enclosure 300. Support structure 250 and enclosure 300 may be composed of a biocompatible material, while other features of cell growth device 100 may be composed of materials selected for structural support over biocompatibility. Furthermore, while various components and features of the present device are shown as continuous or separate pieces, it should be understood that any features of cell growth device 100 may be configured to be the same piece or multiple, separable pieces so long as they still function according to the present disclosure. For example, support structure 250 may be separable from the rest of supporting device 200. Supporting device 200 and enclosure 300 may be reusable and support structure 250 may be disposable. Supporting device 200 and enclosure 300 may be configured to be one continuous piece. Reservoirs 211 A, B may be one, continuous reservoir or multiple discrete reservoirs.

In summary, a device for growing an organoid is provided. A number of cells may be positioned between support structure 250 and enclosure 300, and the cells may then grow on or around support structure 250 to form a 3D organoid. Support structure 250 comprises a number of openings 260 configured to allow the passage of a fluid 400 to the organoid 500 from the interior of the support structure 250. Support structure 250 may also be coupled to a number of reservoirs 211 A, B to facilitate fluid flow through support structure 250. The resulting organoid is hollow and three dimensional.

### Examples

### Tubular Brain Organoid Device Design and Fabrication

The tubular brain organoid device was designed in AutoCAD software. The device was then printed using a stereolithography 3D printer (Form 3B, Formlabs) with a layer thickness of 25 μm.

### Human Embryonic Stem Cell Culture

Human embryonic stem cell WA01 (WiCell) was maintained on Matrigel (Corning) coated 6 well plates in mTESR plus medium (Stemcell Technologies) with a medium change every other day. The WA01 cells were passaged every 7 days using ReLeSR (Stemcell Technologies). WA01 cells under the passage of 42 were used to fabricate brain organoids.

### Fabrication of Human Forebrain and Cerebral Organoids

Human forebrain and cerebral organoids were fabricated. Briefly, 9,000 WA01 cells were harvested from culture and aggregated into embryonic bodies using Aggrewell-800 plates (Stemcell Technologies) in EB formation medium (Stemcell Technologies) supplemented with 10 μM Y-27632 (SelleckChem). The aggregated WA01 cells form EBs in one day. The EBs were then derived into human cerebral organoids using the STEMdiff Cerebral organoid kit (Stemcell Technologies) or derived into human forebrain organoids by dual-SMAD inhibition (Dorsomorhin and A83-01) followed by Wnt activation (CHIR99021) and TGF-beta inhibition (SB431542).

### N2A and HMC3 Cell Culture

Neuro-2a neuroblast cells (N2A) (ATCC) were maintained in Dulbecco's Modified Eagle's Medium (DMEM) (Gibco) supplemented with 10% fetal bovine serum (FBS) (Gibco) and 100 U/mL Penicillin-Streptomycin (Gibco). HMC3 human microglia cells (ATCC) were maintained in DMEM supplemented with 10% FBS and 1× GlutaMAX (Gibco) and 100 U/mL Penicillin-Streptomycin (Gibco). Cells were maintained in a 37° C., 5% CO2 supplemented humidified incubator (Thermo Fisher) and passaged with trypsin-EDTA (Gibco) at 70% confluency.

### Tubular Organoid Fabrication

To fabricate tubular brain organoids, EBs of 3 days were removed from Aggrewell and let sediment in a 15 ml centrifuge tube. Once aggregated at the bottom of the tube, EBs were carefully aspirated using a P1000 pipette with a cupped tip and slowly loaded onto a tubular device. Once EBs fully sedimented inside the device, 50 cL of medium was carefully loaded into both of the side chambers, the device was then transferred to a 6-well plate and carefully topped with an additional 2 mL of the corresponding medium. After EB merge to form a tubular organoid, the culture medium was removed from the device. 50 μL of Matrigel (Corning) was loaded into the center chamber and allowed to solidify in a 37° C. incubator for 30 minutes before loaded with the pre-warmed medium. The device was then cultured for additional 7 days with a medium change every other day before it was placed onto a rocking platform (Thermo Scientific) set at a 7° rocking angle and speed of 1 rpm for continuous rocking inside an incubator. Medium change was performed every other day to maintain the tubular organoids. All medium composition and medium change timeline followed exact protocols used to generate conventional cerebral organoids or forebrain organoids.

### Live/Dead Staining

To visualize cell viability, tubular/conventional brain organoids were subject to staining using live/dead viability kit (Invitrogen). Briefly, samples were incubated with a live/dead staining mix containing CFSE and ethidium homodimer-1. After incubation at 37° C. for 1 hour, the samples were washed twitch with the fresh medium before visualized them on an inverted fluorescence microscope (Olympus IX-83).

### Hypoxia Staining

To visualize hypoxia core formation within tubular/conventional brain organoids, samples were subject to staining by Image-iT red hypoxia kit (Invitrogen). Briefly, samples were incubated with the Image-iT red hypoxia dye for 4 hours before imaged on an inverted fluorescence microscope (Olympus IX-83).

### Cryosection of Organoids

To section the tubular/conventional organoids, the culture samples were first washed twice with 1×PBS (Gibco) and then submerged in 4% paraformaldehyde in PBS (Thermo Scientific) at 4° C. overnight. The samples were then washed twice with 1×PBS and then submerged in 15% sucrose (w/v) for 8 hours followed by 30% sucrose (w/v) overnight at 4° C. to cryoprotect. The organoids were then incubated with 7.5% gelatin (w/v) 10% sucrose (w/v) in PBS solution at 37° C. for 1 hour. Finally, the samples were transferred to a cryomold (Sakura Finetek) and snap-frozen in a dry ice/ethanol slurry. The frozen block was then sectioned on a cryostat (Leica) at 30 μm thickness.

### Immunofluorescence Staining

To characterize the tubular/conventional brain organoid cultures, sectioned samples were placed onto a charged glass slide and washed twice with 1×PBS. The samples were then treated with 3N hydrochloric acid (HCl) for 15 minutes for antigen retrieval. Following HCl treatment, the samples were washed again twice with 1×PBS and subjected to blocking (0.3% Triton-X100, 5% normal goat serum in 1×PBS) for 1 hour, followed by primary antibody incubation in a humidified chamber at 4° C. overnight. The samples were then washed 3 times with 1×PBS followed by secondary antibody at room temperature for 1 hour before washed and coverslipped with gold anti-fade mounting medium with DAPI (Invitrogen). Detailed antibody information and dilution factors can be found in Supplementary Table 51.

### EdU Staining

To characterize neural progenitor cell proliferation, tubular/conventional organoids were stained by Click-iT cell proliferation kit, Alexa 647 (Thermo Fisher). Briefly, organoids were incubated with EdU substrate for 4 hours at 37° C. The samples were then fixed and stained for EdU detection. Following the EdU staining, immunofluorescence staining for PAX6/MAP2 was performed to label neural progenitor cells and neurons as described above.

qPCR Analysis

To analyze the gene expression profile of EBs, brain organoids, and tubular organoids, samples were first washed twice with 1×PBS and lysed for RNA extraction using RNeasy Plus Mini Kit (Qiagen). RNA was then reverse transcribed into cDNA using qScript cDNA synthesis kit (Quantabio). Then cDNA was analyzed by real-time qPCR using SYBR Green real-time PCR master mix (Thermo Fisher). qPCR primer sequences can be found in Supplementary Table S2. Relative expression (−ΔΔCT) was calculated as the delta Ct value between target gene Ct value in organoid and the target gene Ct value in day 1 EB, both normalized against housekeeping gene GAPDH Ct value (ΔΔCT method). Each reaction was triplicated and 5 organoids/EB s were used for each group. Mean Ct value was used. Samples that did not amplify for a certain gene were denoted with Ct values of 40.

Microglia Differentiation from WA01 Cells

To differentiate WA01 cells into microglia, WA01 cells were plated onto Matrigel (Corning) coated 6-well plate at a density of 30-40 colonies per well. The WA01 cells were then differentiated into hematopoietic progenitor cells (HPC) using STEMdiff hematopoietic kit (Stemcell Technologies). The differentiated WA01 derived HPC was then further differentiated into induced microglia (iMG) cells using STEMdiff Microglia Differentiation kit (Stemcell Technologies).

Microglia Treatment with Drugs

To treat microglia cultured in well plates or microglia embedded in hFO, lipopolysaccharide (LPS) (Sigma) is dosed at 100 ng/mL for 24 hours followed by Adenosine 5′-triphosphate (ATP) (Sigma) at a concentration of 2 mM for 30 minutes. Similarly, DAMGO was given at a concentration of 100 nM for 24 hours. For LY-2828360 (Cayman Chemicals) treatment, microglia treated with DAMGO for 24 hours was refreshed with a medium containing 100 nM LY-2828360 for an additional 24 hours.

### Inflammasome NLRP3/ASC Colocalization Proximity Ligation Assay

To visualize inflammasome activation inside microglia cells, we analyzed NLRP3/ASC colocalization by proximity ligation assay (PLA). Samples were subject to NLRP3/ASC staining following by colocalization PLA using the Duolink in situ red kit (Sigma). After the PLA labeling, samples were then additionally labeled by Iba1/MAP2 before coverslipped with Prolong gold anti-fade DAPI mounting medium (Thermo Fisher) and visualized under an inverted fluorescence microscope (Olympus IX-83).

### ELISA Assays

Conditioned medium of 24 hours (total volume 500 μL) from control conditions or drug-treated conditions were collected from microglia cultured in 24 well plates or incorporated inside tubular organoids. The medium was centrifuged at 2,000 g for 10 minutes to remove cell debris. 400 μL of supernatant was then aspirated and used for ELISA assays. Samples were assayed using ELISA plates for IL-1β (Tribioscience), IL-18 (Tribioscience), and TNF-α (Abcam). Each sample was tested in triplicates and read at 450 nm for absorbance.

### Statistical Analysis

The statistics comparing two sample groups were conducted using the students' t-test. Statistical significance was denoted as following: *p<0.05, **p<0.01, ***p<0.005. ****p<0.001.

### Results and Discussion

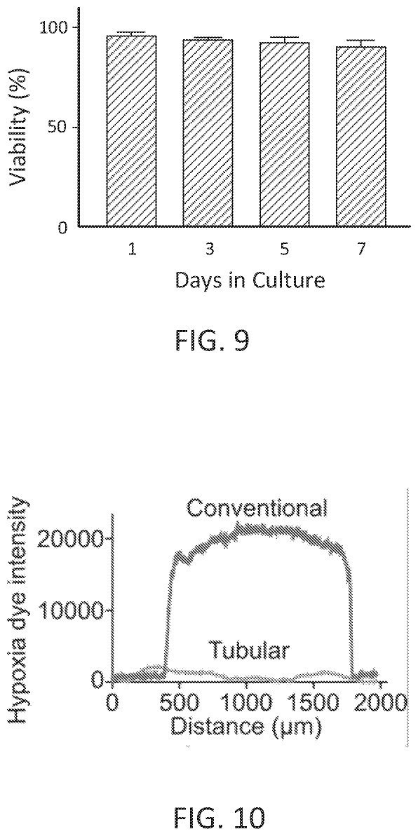

Referring to FIGS. 8 and 9, to characterize on-chip 3D culture and cell viability, Neuro-2A (N2A) mouse neuroblast spheroids were first cultured as control and then loaded into the loading basket of our tubular device. The spheroids were fabricated with a 300 μm average diameter so that the loading basket with 250 μm perfusable gaps could hold the spheroids without them leaking out of the device. Over 2 days in culture, the spheroids merged to form a tubular shape. A sectioned the tubular N2a culture was fixed to confirm the formation of tubular-shaped structure. To confirm that the tubular brain organoid device could maintain high cell viability, these tubular N2a cultures were then characterized for viability. As expected, the tubular 3D culture showed good viability over prolonged culture time (See FIG. 9). As we confirmed the high viability using N2A 3D culture, we further characterized the hypoxic core formation inside our tubular 3D culture versus traditional organoid culture using the hypoxia indicator dye. We found that the tubular 3D cultures of N2A cells are hypoxia-free, as sufficient oxygen can be provided by perfusion from both the inner lumen and outside skirt gaps (See FIG. 10).

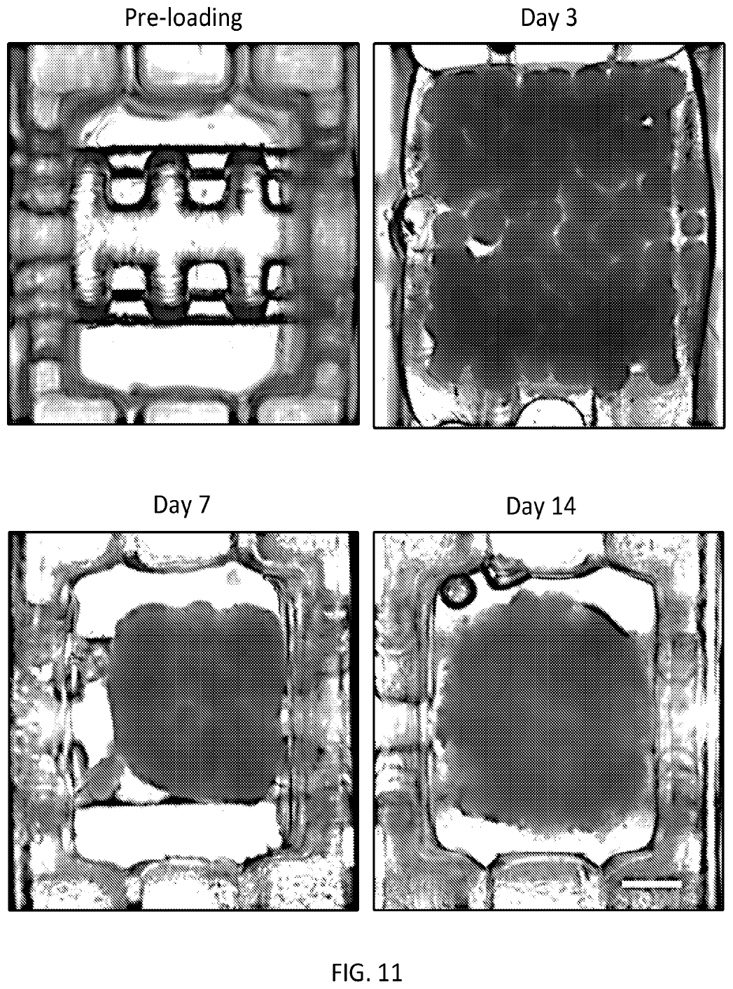

Referring to FIG. 10, To fabricate tubular organoids using our device, we adapted a human forebrain organoid fabrication protocol. Briefly, EB fabricated from WA01 human pluripotent stem cells (hPSC) of 300 μm in size were loaded into the loading basket at day 3, loaded with medium containing dual-SMAD inhibitors dorsomorphin and A83 for neural induction. The EBs were cultured inside the tubular organoid device and neural induction medium for 4 additional days to allow for EB merging to form a tubular culture surrounding the inner lumen. As shown in FIG. 11, after the tubular culture was formed, we then loaded the inner basket with Matrigel to embed the organoid. The medium was then switched to CHIR-99021 and SB-431542 containing medium to activate the Wnt signaling pathway and guide its forebrain identity. The organoids were cultured for 7 additional days inside the inner basket for neural epithelium expansion. The device was finally loaded onto a rocking platform to create inner lumen fluid flow for medium perfusion and mechanical stimulation, with N2, B27 supplemented medium to support neuron growth.

In summary, a hollow human brain organoid culture to model neural inflammation is disclosed. The hollow organoid culture eliminated hypoxic core formation by scaffold guided growth and rocking-platform induced constant passive flow perfusion. The hollow organoid culture showed reduced heterogeneity and improved neural development. Additionally, it was demonstrated that the incorporation of isogenic microglia cells with non-invasive flow introduction, preserving its homeostatic morphology and function. Moreover, the iMG incorporated into the hollow organoid culture can respond to external stimulation such as LPS and DAMGO, recapitulating the in vivo phenotype and functions during neural inflammation. The neural inflammation model can also be subject to therapeutics screening as evidenced by its evident responses to LY282860 treatment.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

All publications, patents and patent applications referenced herein are hereby incorporated by reference in their entirety for all purposes as if each such publication, patent or patent application had been individually indicated to be incorporated by reference.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A device for supporting growth of a three dimensional tubular organoid, the device comprising:
a support structure having a wall and a lumen extending from a first end to a second end, wherein the wall comprises a plurality of openings extending therethrough;
an enclosure configured to extend at least partially around the support structure and support a plurality of cells disposed between the enclosure and the support structure; and
a first reservoir fluidly coupled to at least one of the ends of the support structure.

2. The device of claim 1, wherein the enclosure is configured to be removed from extending at least partially around the support structure.

3. The device of claim 1, wherein the plurality of openings comprise at least one of holes, pores, slots, mesh, and semi-permeable membranes.

4. The device of claim 1, wherein the plurality of openings have a size from about 1 micron to about 1 cm; and optionally, wherein the plurality of openings have a size of about 250 micron.

5. The device of claim 1, wherein the wall has a size from about 0.5 microns to about 2 millimeters; and optionally, wherein the wall has a size of about 1 micron to about 500 microns.

6. The device of claim 1, wherein the support structure is formed from an additive manufacturing process, or wherein the support structure is composed of a biocompatible polymer.

7. The device of claim 1, further comprising a second reservoir, wherein the first reservoir is fluidly coupled to the first end of the support structure and the second reservoir is fluidly coupled to the second end of the support structure; and optionally, further comprising a fluid agent within the first and second reservoirs, the fluid comprising at least one of a medium, cells, and therapeutic agents; and optionally, wherein the fluid agent is configured to contact at least a portion of the 3D organoid through the permeable features.

8. The device of claim 1, wherein the system is configured to be positioned within a standard well plate.

9. The device of claim 1, wherein the plurality of cells comprise a group selected from a cell mass, an embryonic body, a tissue fragment, and a cell culture.

10. A method of growing a three dimensional hollow organoid comprising:
providing a device comprising:
a support structure having a wall and a lumen extending from a first end to a second end of the lumen, wherein the wall comprises a plurality of openings extending therethrough;
an enclosure configured to extend at least partially around the support structure and support a plurality of cells disposed between the enclosure and the support structure;
a first reservoir fluidly coupled to the first end of the support structure; and
a second reservoir fluidly coupled to the second end of the support structure;
positioning the plurality of cells between the support structure and the enclosure; and
providing at least one fluid into the first reservoir or the second reservoir and allowing the at least one fluid to flow into the lumen of the support structure and through the openings of the support structure, wherein the at least one fluid contacts the cells, thereby facilitating growth of the cells into a three dimensional hollow organoid surrounding the support structure.

11. The method of claim 10, further comprising the step of treating the three dimensional organoid with an active agent.

12. The method of claim 10, further comprising rocking the device about an axis perpendicular to a length of the support structure, wherein the length is defined to be from the first end to the second end of the support structure.

13. The method of claim 10, wherein the device is composed of a biocompatible polymer.

14. The method of claim 10, wherein the support structure is tubular.

15. A method of growing a three dimensional hollow organoid comprising:
providing a device comprising:
a support structure having a wall and a lumen extending from a first end to a second end of the lumen, wherein the wall comprises a plurality of openings extending therethrough;
an enclosure configured to extend at least partially around the support structure and support a plurality of cells, a cell mass, or a tissue fragment disposed between the enclosure and the support structure;
a first reservoir fluidly coupled to the first end of the support structure; and
a second reservoir fluidly coupled to the second end of the support structure;
positioning the plurality of cells between the support structure and the enclosure; and
providing at least one fluid into the first reservoir or the second reservoir and allowing the at least one fluid to flow into the lumen of the support structure and through the openings of the support structure, wherein the at least one fluid contacts the cells, thereby facilitating growth of the cells into a three dimensional hollow organoid surrounding the support structure.
